(19) United States
(12) Patent Application Publication
(10) Pub. No.: US 2005/0059807 A1
(43) Pub. Date: Mar. 17, 2005

(54) Novel polypeptide-transcription factor xap-2-protein 49.72 and the polynucleotide encoding said polypeptide

(76) Inventors: Yumin Mao, Shanghai (CN); Yi Xie, Shanghai (CN)

Correspondence Address: COHEN, PONTANI, LIEBERMAN & PAVANE, 551 FIFTH AVENUE, SUITE 1210, NEW YORK, NY, 10176, US

(21) Appl. No.: 10/363,870

(22) PCT Filed: Sep. 10, 2001
(86) PCT No.: PCT/CN01/01346

(30) Foreign Application Priority Data
Sep. 12, 2000 (CN) 00125185.6

(51) Int. Cl. C07H 21/04; C07K 14/475

(52) U.S. Cl. 530/399; 536/23.5; 435/69.1; 435/320.1; 435/325

(57) ABSTRACT

The invention discloses a new polypeptide-transcription factor XAP-2 protein 49.72, the polynucleotide encoding said polypeptide, and a process for producing the polypeptide by recombinant DNA technology, It also discloses methods of applying the polypeptide for the treatment of various diseases, such as fetation abnormality, tumor, diabetes, catamenia disharmony, peptic ulcer, arhythmia, anaemia, epilepsia. This invention also discloses antagonist against said polypeptide and thereof therapy uses. In addition, it refers to the use of said polynucleotide encoding this new transcription factor XAP-2 protein 49.72.

### FIELD OF THE INVENTION

The invention relates to the field of biotechnology. In particular, the invention relates to a novel polypeptide, transcription factor XAP-2 protein 49.72, and a polynucleotide sequence encoding said polypeptide. The invention also relates to the method for the preparation and use of said polynucleotide and polypeptide.

### TECHNICAL BACKGROUND

There are very few transcription factors that are regulated in the process of embryogenesis. In Xenopus, most of the transcription factors are responsible for the expression of genes from the very beginning of the embryo transcription, the metaphase blastula transition stage. These transcription factors are thought to be derived from oocyt synthesized maternal RNAs because they are active before the embryo transcription.

AP-2 is a transcription factor involved in development transcriptions. AP-2 binding site was found in many human protein coding genes like growth hormone, proenkaphalin and collagenase. A common conservative sequence, 5′-CCCCAGGC-3′ (Mitchell, P. J., Cell50,2 1359-260,1987), was found through the comparison of these binding site sequences. A transient expression promoting function of AP-2 exists in the differentiation process of abnormal causing cancer cells induced by retinoic acid (RA) (Luscher, B., Genes Dev. 3,1507-1517,1989). RA is involved in the formation of the limbs of vertebrate, so AP-2 plays an important role in regulating gene expression of embryo early development

Through Northern blotting analysis, we find that there are three transcription forms of XAP-2(Xenopus AP-2), 1.8 kb, 2.2 kb, 2.6 kb, while two in human AP-2, 3.3 kb and 3.6 kb. There is no expression of Xenopus AP-2 at the time of oogenesis. All of them play their role in the ninth stage after blastula transition, but with different function and tissue specificity. Thus it can be concluded that Xenopus AP-2 has more than one function in Xenopus embryo genesis.

Xenopus AP-2 gene has 1573 bps, with an open reading frame coding for 425 amino acid (Williams, T., Genes. Dev. 2,1557-1569,1988). The Xenopus AP-2 gene shares 88.1% identity and 92.7% similarity with human AP-2 gene.

Human AP-2 gene expression is promoted by retinoic acid. But there is no obvious promoting function of retinoic acid to Xenopus AP-2 gene. AP-2 plays an important role in the transcription regulation of the retinoic acid affected morphologic development (Mitchell. P. J., Genes Dev. 5, 105-119,1991).

The polypeptide in this invention shares 55% identity and 66% similarity with Xenopus AP-2. It also contains the characteristic conservative sequence of AP family. Therefore this protein is deduced as a new protein classification correlated protein and has similar biologic function. This protein is named XAP-2 protein 49.72.

As discussed, transcription factor XAP-2 protein 49.72 plays an essential role in the regulation of important biological functions such as cell division and embryogenesis, and it's believed that numerous proteins are involved in these regulations. So the identification of the transcription factor XAP-2 protein 49.72, especially its amino acid sequence, is always desired in this filed. The isolation of this novel transcription factor XAP-2 protein 49.72 builds the basis for research of the protein function under normal and clinical conditions, disease diagnosis and drug development. So the ioslation of its cDNA is very improtant.

### DESCRIPTION OF THE INVENTION

One objective of the invention is to provide an isolated novel polypeptide, i.e., a transcription factor XAP-2 protein 49.72, and fragments, analogues and derivatives thereof.

Another objective of the invention is to provide a polynucleotide encoding said polypeptide.

Another objective of the invention is to provide a recombinant vector containing a polynucleotide encoding a transcription factor XAP-2 protein 49.72.

Another objective of the invention is to provide a genetically engineered host cell containing a polynucleotide encoding a transcription factor XAP-2 protein 49.72.

Another objective of the invention is to provide a method for producing a transcription factor XAP-2 protein 49.72.

Another objective of the invention is to provide an antibody against a transcription factor XAP-2 protein 49.72 of the invention.

Another objective of the invention is to provide mimetics, antagonists, agonists, and inhibitors for the polypeptide of the transcription factor XAP-2 protein 49.72.

Another objective of the invention is to provide a method for the diagnosis and treatment of the diseases associated with an abnormality of transcription factor XAP-2 protein 49.72.

### SUMMARY OF INVENTION

The present invention relates to an isolated polypeptide, which is originated from human, and comprises a polypeptide having the amino acid sequence of <210> 2, or its conservative variants, or its active fragments, or its active derivatives and its analogues. Preferably, the polypeptide has the amino acid sequence of <210> 2.

The present invention also relates to an isolated polynucleotide, comprising a nucleotide sequence or its variant selected from the group consisting of (a) the polynucleotide encoding a polypeptide comprising the amino acid sequence of <210> 2; and (b) a polynucleotide complementary to the polynucleotide (a); (c) a polynucleotide that shares at least 70% homology to the polynucleotide (a) or (b). Preferably, said nucleotide sequence is selected from the group consisting of (a) the sequence of position 216-1574 in <210> 1; and (b) the sequence of position 1-1778 in <210> 1.

The invention also includes: a vector containing a polynucleotide of said invention, especially an expression vector; a host cell genetically engineered with the vector via transformation, transduction or transfection; a method for the production of said inventive polypeptide through the process of host cell cultivation and expression product harvest.

The invention also relates to an antibody which specifically binds to the inventive polypeptide.

The invention also relates to a method for selecting compounds which could simulate, activate, antagonize, or inhibit the activity of the inventive polypeptide and the compounds obtained by the method.

The invention also relates to a method for in vitro diagnosis method of the diseases or disease susceptibility related with the abnormal expression of the inventive polypeptide. The method involves the detection of mutation in the polypeptide or its encoding polynucleotide sequence, or the determination of its quantity and/or biological activity in biological samples.

The invention also relates to pharmaceutical compositions which comprise the inventive polypeptide, its analogues, mimetics, agonists, antagonists, inhibitors, and a pharmaceutically acceptable carrier.

The invention also relates to applications of the inventive polypeptide and/or its polynucleotide for drug development to treat cancers, developmental diseases, immune diseases, or other diseases caused by abnormal expression of the inventive polypeptide.

Other aspects of the invention are apparent to the skilled in the art in view of the disclosure set forth hereinbelow.

### BRIEF DESCRIPTION OF THE DRAWINGS

The following drawings are provided to illustrate the embodiment of the invention, not to limit the scope of invention defined by the claims.

FIG. 1 shows an alignment comparison of amino acid sequences of transcription factor XAP-2 protein 49.72 of the invention and Transcription factor XAP-2 protein. The upper sequence is transcription factor XAP-2 protein 49.72, and the lower sequence is Transcription factor XAP-2 protein. The identical and similar amino acids are indicated by a one-letter code of amino acid and “+” respectively.

FIG. 2 shows the SDS-PAGE of the isolated transcription factor XAP-2 protein 49.72, which has a molecular weight of 49.72 kDa. The isolated protein band is marked with an arrow.

### DESCRIPTION OF INVENTION

The terms used in this specification and claims have the following meanings, unless otherwise noted.

“Nucleotide sequence” refers to oligonucleotide, nucleotide, or polynucleotide and parts of polynucleotide. It also refers to genomic or synthetic DNA or RNA, which could be single stranded or double stranded, and could represent the sense strand or the antisense strand. Similarly, the term “amino acid sequence” refers to oligopeptide, peptide, polypeptide, or protein sequence and parts of proteins. When the “amino acid sequence” in the invention is related to the sequence of a natural protein, the amino acid sequence of said “peptide” or “protein” will not be limited to be identical to the sequence of that natural protein.

“Variant” of a protein or polynucleotide refers to the amino acid sequence or nucleotide sequence, respectively with one or more amino acids or one or more nucleotides changed. Such changes include deletion, insertion, and/or substitution of amino acids in the animo acid sequence, or of nucleotides in the polynucleotide sequence. These changes could be conservative and the substituted amino acid has similar structural or chemical characteristics as the original one, such as the substitution of Ile with Leu. Changes also could be not conservative, such as the substitution of Ala with Trp.

“Deletion” refers to the deletion of one or several amino acids in the amino acid sequence, or of one or several nucleotides in the nucleotide sequence.

“Insertion” or “addition” refers to the addition of one or several amino acids in the amino acid sequence, or of one or several nucleotides in the nucleotide sequence, comparing to the natural molecule. “Substitution” refers to the change of one or several amino acids, or of one or several nucleotides, into different ones without changing number of the residues.

“Biological activity” refers to structural, regulatory or biochemical characteristics of a natural molecule. Similarly, the term “immungenecity” refers to the ability of natural, recombinant, or synthetic proteins to inducing a specific immunologic reaction, or of binding specific antibody in appropriate kind of animal or cell.

“Agonist” refers to molecules which regulate, but generally enhance the activity of the inventive polypeptide by binding and changing it. Agonists include proteins, nucleotides, carbohydrates or any other molecules which could bind the inventive polypeptide.

“Antagonist” or “inhibitor” refers to molecules which inhibit or downregulate the biological activity or immunogenecity the inventive polypeptide via binding to it. Antagonists or inhibitors include proteins, nucleotides, carbohydrates or any other molecules which bind to the inventive polypeptide.

“Regulation” refers to changes in function of the inventive polypeptide, including up-regulation or down-regulation of the protein activity, changes in binding specifity, changes of any other biological characteristics, functional or immune characteristics.

“Substantially pure” refers to the condition of substantially free of other naturally related proteins, lipids, saccharides, or other substances. One of ordinary skill in the art can purify the inventive polypeptide by standard protein purification techniques. Substantially pure polypeptide of the invention produces a single main band in a denaturing polyacrylamide gel. The purity of a polypeptide may also be analyzed by amino acid sequence analysis.

“Complementary” or “complementation” refers to the binding of polynucleotides by base pairing under the condition of approximate ion conditions and temperature. For instance, the sequence “C-T-G-A” could bind its complementary sequence “G-A-C-T.” The complementation between two single strand molecules could be partial or complete. Homology or sequence similarity between two single strands obviously influences the efficiency and strength of the formed hybrid.

“Homology” refers to the complementary degree, which may be partially or completely homologous. “Partial homology” refers to a sequence being partially complementary to a target nucleotide. The sequence could at least partially inhibit the hybridization between a completely complementary sequence and the target nucleotide. Inhibition of hybridization could be assayed by hybridization (Southern blot or Northern blot) under less stringent conditions. Substantially complementary sequence or hybrid probe could compete with the completely complementary sequence and inhibit its hybridization with the target sequence under less stringent conditions. This doesn't mean that nonspecific binding is allowed under a less stringent condition, because specific or selective reaction is still required.

“Sequence Identity” refers to the percentage of sequence identity or similarity when two or several amino acid or nucleotide sequences are compared. Sequence identity may be determined by computer programs such as MEGALIGN (Lasergene Software Package, DNASTAR, Inc., Madison Wis.). MEGALIGN can compare two or several sequences using different methodologies such as the Cluster method (Higgins, D. G. and P. M. Sharp (1988) Gene 73: 237-244). Cluster method examines the distance between all pairs and arrange the sequences into clusters. Then the clusters are partitioned by pair or group. The sequence identity between two amino acid sequences such as sequence A and B can be calculated by the following equation:

$$\frac{( \begin{matrix}\text{Number of paired identical residues} \\ \text{between sequences A and B}\end{matrix} )}{( \begin{matrix}\text{Residue number of sequence A} - \\ \text{number of gap residues in sequence A} - \\ \text{number of gap residue in sequence B}\end{matrix} )} \times 100$$

Sequence identity between nucleotide sequences can also be determined by Cluster method or other well-known methods in the art such as the Jotun Hein method (Hein J., (1990) Methods in Emzymology 183: 625-645)

“Similarity” refers to the degree of identity or conservative substitution degree of amino acid residues in corresponding sites of the amino acid sequences when compared to each other. Amino acids for conservative substitution are: negative charged amino acids including Asp and Glu; positive charged amino acids including Leu, Ile and Val; Gly and Ala; Asn and Gln; Ser and Thr; Phe and Tyr.

“Antisense” refers to the nucleotide sequences complementary to a specific DNA or RNA sequence. “Antisense strand” refers to the nucleotide strand complementary to the “sense strand.”

“Derivative” refers to the inventive polypeptide or the chemically modified nucleotide encoding it. This kind of modified chemical can be derived from replacement of the hydrogen atom with Alkyl, Acyl, or Amino. The nucleotide derivative can encode peptide retaining the major biological characteristics of the natural molecule.

“Antibody” refers to the intact antibody or its fragments such as Fa, F(ab′)₂and Fv, and it can specifically bind to antigenic epitopes of the inventive polypeptide.

“Humanized antibody” refers to an antibody which has its amino acid sequence in non-antigen binding region replaced to mimic human antibody and still retain the original binding activity.

The term “isolated” refers to the removal of a material out of its original environment (for instance, if it's naturally produced, original environment refers to its natural environment). For example, a naturally produced polynucleotide or a polypeptide in its original host organism means it has not been “isolated,” while the separation of the polynucleotide or a polypeptide from its coexisting materials in natural system means it was “isolated.” This polynucleotide may be a part of a vector, or a part of a compound. Since the vector or compound is not part of its natural environment, the polynucleotide or peptide is still “isolated.”

As used herein, the term “isolated” refers to a substance which has been isolated from the original environment. For naturally occurring substance, the original environment is the natural environment. For example, the polynucleotide and polypeptide in a naturally occurring state in the viable cells are not isolated or purified. However, if the same polynucleotide and polypeptide have been isolated from other components naturally accompanying them, they are isolated or purified.

As used herein, “isolated transcription factor XAP-2 protein 49.72,” means that transcription factor XAP-2 protein 49.72 does not essentially contain other proteins, lipids, carbohydrate or any other substances associated therewith in nature. The skilled in the art can purify transcription factor XAP-2 protein 49.72, by standard protein purification techniques. The purified polypeptide forms a single main band on a non-reducing PAGE gel. The purity of transcription factor XAP-2 protein 49.72 can also be analyzed by amino acid sequence analysis.

The invention provides a novel polypeptide—transcription factor XAP-2 protein 49.72, which comprises the amino acid sequence shown in <210> 2. The polypeptide of the invention may be a recombinant polypeptide, natural polypeptide, or synthetic polypeptide, preferably a recombinant polypeptide. The polypeptide of the invention may be a purified natural product or a chemically synthetic product. Alternatively, it may be produced from prokaryotic or eukaryotic hosts, such as bacterial, yeast, higher plant, insect, and mammal cells, using recombinant techniques. Depending on the host used in the protocol of recombinant production, the polypeptide of the invention may be glycosylated or non-glycosylated. The polypeptide of the invention may or may not comprise the starting Met residue.

The invention further comprises fragments, derivatives and analogues of transcription factor XAP-2 protein 49.72. As used in the invention, the terms “fragment,” “derivative” and “analogue” mean the polypeptide that essentially retains the same biological functions or activity of transcription factor XAP-2 protein 49.72 of the invention. The fragment, derivative or analogue of the polypeptide of the invention may be (i) one in which one or more of the amino acid residues are substituted with a conserved or non-conserved amino acid residue (preferably a conserved amino acid residue) and such substituted amino acid residue may or may not be one encoded by the genetic code; or (ii) one in which one or more of the amino acid residues are substituted with other residues, including a substituent group; or (iii) one in which the mature polypeptide is fused with another compound, such as a compound to increase the half-life of the polypeptide (for example, polyethylene glycol); or (iv) one in which additional amino acids are fused to the mature polypeptide, such as a leader or secretory sequence or a sequence which is employed for purification of the mature polypeptide or a proteinogen sequence. Such fragments, derivatives and analogs are deemed to be within the scope of the skilled in the art from the teachings herein.

The invention provides an isolated nucleic acid or polynucleotide which comprises the polynucleotide encoding an amino acid sequence of <210> 2. The polynucleotide sequence of the invention includes the nucleotide sequence of <210> 1. The polynucleotide of the invention was identified in a human embryonic brain cDNA library. Preferably, it comprises a full-length polynucleotide sequence of 1778 bp, whose ORF (216-1574) encodes 452 amino acids. Based on amino acid homology comparison, it is found that the encoded polypeptide is 55% homologous to Transcription factor XAP-2 protein. This novel Transcription factor XAP-2 protein 49.72 has similar structures and biological functions to those of Transcription factor XAP-2 protein.

The polynucleotide according to the invention may be in the forms of DNA or RNA. The forms of DNA include cDNA, genomic DNA, and synthetic DNA, etc., in single stranded or double stranded form. DNA may be an encoding strand or a non-encoding strand. The coding sequence for mature polypeptide may be identical to the coding sequence shown in <210> 1, or is a degenerate sequence. As used herein, the term “degenerate sequence” means a sequence which encodes a protein or peptide comprising a sequence of <210> 2 and which has a nucleotide sequence different from the sequence of coding region in <210> 1.

The polynucleotide encoding the mature polypeptide of <210> 2 includes those encoding only the mature polypeptide, those encoding mature polypeptide plus various additional coding sequence, the coding sequence for mature polypeptide (and optional additional encoding sequence) plus the non-coding sequence.

The term “polynucleotide encoding the polypeptide” includes polynucleotides encoding said polypeptide and polynucleotides comprising additional coding and/or non-coding sequences.

The invention further relates to variants of the above polynucleotides which encode a polypeptide having the same amino acid sequence of invention, or a fragment, analogue and derivative of said polypeptide. The variant of the polynucleotide may be a naturally occurring allelic variant or a non-naturally occurring variant. Such nucleotide variants include substitution, deletion, and insertion variants. As known in the art, an allelic variant may have a substitution, deletion, and insertion of one or more nucleotides without substantially changing the functions of the encoded polypeptide.

The present invention further relates to polynucleotides, which hybridize to the hereinabove-described sequences, that is, there is at least 50% and preferably at least 70% identity between the sequences. The present invention particularly relates to polynucleotides which hybridize to the polynucleotides of the invention under stringent conditions. As herein used, the term “stringent conditions” means the following conditions: (1) hybridization and washing under low ionic strength and high temperature, such as 0.2×SSC, 0.1% SDS, 60° C.; or (2) hybridization after adding denaturants, such as 50% (v/v) formamide, 0.1% bovine serum/0.1% Ficoll, 42° C.; or (3) hybridization only when the homology of two sequences at least 95%, preferably 97%. Further, the polynucleotides which hybridize to the hereinabove described polynucleotides encode a polypeptide which retains the same biological function and activity as the mature polypeptide of <210> 2

The invention also relates to nucleic acid fragments hybridized with the hereinabove sequence. As used in the present invention, the length of the “nucleic acid fragment” is at least more than 10 bp, preferably at least 20-30 bp, more preferably at least 50-60 bp, and most preferably at least 100 bp. The nucleic acid fragment can be used in amplification techniques of nucleic acid, such as PCR, so as to determine and/or isolate the polynucleotide encoding transcription factor XAP-2 protein 49.72.

The polypeptide and polynucleotide of the invention are preferably in the isolated form, preferably purified to be homogenous.

According to the invention, the specific nucleic acid sequence encoding transcription factor XAP-2 protein 49.72 can be obtained in various ways. For example, the polynucleotide is isolated by hybridization techniques well-known in the art, which include, but are not limited to 1) the hybridization between a probe and genomic or cDNA library so as to select a homologous polynucleotide sequence, and 2) antibody screening of expression library so as to obtain polynucleotide fragments encoding polypeptides having common structural features.

According to the invention, DNA fragment sequences may further be obtained by the following methods: 1) isolating double-stranded DNA sequence from genomic DNA; and 2) chemical synthesis of DNA sequence so as to obtain the double-stranded DNA.

Among the above methods, the isolation of genomic DNA is least frequently used. A commonly used method is the direct chemical synthesis of DNA sequence. A more frequently used method is the isolation of cDNA sequence. Standard methods for isolating the cDNA of interest is to isolate mRNA from donor cells that highly express said gene followed by reverse transcription of mRNA to form plasmid or phage cDNA library. There are many established techniques for extracting mRNA and the kits are commercially available (e.g. Qiagene). Conventional method can be used to construct cDNA library (Sambrook, et al., Molecular Cloning, A Laboratory Manual, Cold Spring Harbor Laboratory. New York, 1989). The cDNA libraries are also commercially available. For example, Clontech Ltd. has various cDNA libraries. When PCR is further used, even an extremely small amount of expression products can be cloned.

Numerous well-known methods can be used for screening for the polynucleotide of the invention from cDNA library. These methods include, but are not limited to, (1) DNA-DNA or DNA-RNA hybridization; (2) the appearance or loss of the function of the marker-gene; (3) the determination of the level of transcription factor XAP-2 protein 49.72 transcripts; (4) the determination of protein product of gene expression by immunology methods or the biological activity assays. The above methods can be used alone or in combination.

In method (1), the probe used in the hybridization could be homologous to any portion of polynucleotide of invention. The length of probe is typically at least 10 nucleocides, preferably at least 30 nucleocides, more preferably at least 50 nucleocides, and most preferably at least 100 nucleotides. Furthermore, the length of the probe is usually less than 2000 nucleotides, preferably less than 1000 nucleotides. The probe usually is the DNA sequence chemically synthesized on the basis of the sequence information. Of course, the gene of the invention itself or its fragment can be used as a probe. The labels for DNA probe include, e.g., radioactive isotopes, fluoresceins or enzymes such as alkaline phosphatase.

In method (4), the detection of the protein products expressed by transcription factor XAP-2 protein 49.72 gene can be carried out by immunology methods, such as Western blotting, radioimmunoassay, and ELISA.

The method of amplification of DNA/RNA by PCR (Saiki, et al. Science 1985; 230: 1350-1354) is preferably used to obtain the polynucleotide of the invention. Especially when it is difficult to obtain the full-length cDNA, the method of RACE (RACE-cDNA terminate rapid amplification) is preferably used. The primers used in PCR can be selected according to the polynucleotide sequence information of the invention disclosed herein, and can be synthesized by conventional methods. The amplified DNA/RNA fragments can be isolated and purified by conventional methods such as gel electrophoresis.

Sequencing of polynucleotide sequence of the gene of the invention or its various DNA fragments can be carried out by the conventional dideoxy sequencing method (Sanger et al. PNAS, 1977, 74: 5463-5467). Sequencing of polynucleotide sequence can also be carried out using the commercially available sequencing kits. In order to obtain the full-length cDNA sequence, it is necessary to repeat the sequencing process. Sometimes, it is needed to sequence the cDNA of several clones to obtain the full-length cDNA sequence.

The invention further relates to a vector comprising the polynucleotide of the invention, a genetically engineered host cell transformed with the vector of the invention or directly with the sequence encoding transcription factor XAP-2 protein 49.72, and a method for producing the polypeptide of the invention by recombinant techniques.

In the present invention, the polynucleotide sequences encoding transcription factor XAP-2 protein 49.72 may be inserted into a vector to form a recombinant vector containing the polynucleotide of the invention. The term “vector” refers to a bacterial plasmid, bacteriophage, yeast plasmid, plant virus or mammalian virus such as adenovirus, retrovirus or any other vehicle known in the art. Vectors suitable for use in the present invention include, but are not limited to the T7-based expression vector for expression in bacteria (Rosenberg, et al., Gene, 56: 125, 1987), the pMSXND expression vector for expression in mammalian cells (Lee and Nathans, J Biol. Chem., 263: 3521, 1988) and baculovirus-derived vectors for expression in insect cells. Any plasmid or vector can be used to construct the recombinant expression vector as long as it can replicate and is stable in the host. One important feature of an expression vector is that the expression vector typically contains an origin of replication a promoter, a marker gene as well as translation regulatory components.

Methods known in the art can be used to construct an expression vector containing the DNA sequence of transcription factor XAP-2 protein 49.72 and appropriate transcription/translation regulatory components. These methods include in vitro recombinant DNA technique, DNA synthesis technique, in vivo recombinant technique and so on (Sambroook, et al. Molecular Cloning, a Laboratory Manual, cold Spring Harbor Laboratory. New York, 1989). The DNA sequence is operatively linked to a proper promoter in an expression vector to direct the synthesis of mRNA. Exemplary promoters are lac or trp promoter of E. coli; PL promoter of λ phage; eukaryotic promoters including CMV immediate early promoter, HSV thymidine kinase promoter, early and late SV40 promoter, LTRs of retrovirus, and other known promoters which control gene expression in the prokaryotic cells, eukaryotic cells or viruses. The expression vector may further comprise a ribosome binding site for initiating translation, transcription terminator and the like. Transcription in higher eukaryotes is increased by inserting an enhancer sequence into the vector. Enhancers are cis-acting elements of DNA, usually about from 10 to 300 bp in length that act on a promoter to increase gene transcription level. Examples include the SV40 enhancer on the late side of the replication origin 100 to 270 bp, the polyoma enhancer on the late side of the replication origin, and adenovirus enhancers.

Further, the expression vector preferably comprises one or more selective marker genes to provide a phenotype for the selection of the transformed host cells, e.g., the dehydrofolate reductase, neomycin resistance gene and GFP (green flurencent protein) for eukaryotic cells, as well as tetracycline or ampicillin resistance gene for E. coli.

An ordinarily skilled in the art knows clearly how to select appropriate vectors, transcriptional regulatory elements, e.g., promoters, enhancers, and selective marker genes.

According to the invention, polynucleotide encoding transcription factor XAP-2 protein 49.72 or recombinant vector containing said polynucleotide can be transformed or transfected into host cells to construct genetically engineered host cells containing said polynucleotide or said recombinant vector. The term “host cell” means prokaryote, such as bacteria; or primary eukaryote, such as yeast; or higher eukaryotic, such as mammalian cells. Representative examples are bacterial cells, such as E. coli, Streptomyces, Salmonella typhimurium; fungal cells, such as yeast; plant cells; insect cells such as Drosophila S2 or Sf9; animal cells such as CHO, COS or Bowes melanoma.

Transformation of a host cell with the DNA sequence of invention or a recombinant vector containing said DNA sequence may be carried out by conventional techniques as are well known to those skilled in the art. When the host is prokaryotic, such as E. coli, competent cells, which are capable of DNA uptake, can be prepared from cells harvested after exponential growth phase and subsequently treated by the CaCl₂ method using procedures well known in the art. Alternatively, MgCl₂ can be used. Transformation can also be carried out by electroporation, if desired. When the host is an eukaryote, transfection methods as well as calcium phosphate precipitation may be used. Conventional mechanical procedures such as micro-injection, electroporation, or liposome-mediated transfection may also be used.

The recombinant transcription factor XAP-2 protein 49.72 can be expressed or produced by the conventional recombinant DNA technology (Science, 1984; 224: 1431), using the polynucleotide sequence of the invention. The steps generally include:
(1) transfecting or transforming the appropriate host cells with the polynucleotide (or variant) encoding transcription factor XAP-2 protein 49.72 of the invention or the recombinant expression vector containing said polynucleotide;
(2) culturing the host cells in an appropriate medium; and
(3) isolating or purifying the protein from the medium or cells.

In Step (2) above, depending on the host cells used, the medium for cultivation can be selected from various conventional mediums. The host cells are cultured under a condition suitable for its growth until the host cells grow to an appropriate cell density. Then, the selected promoter is induced by appropriate means (e.g., temperature shift or chemical induction) and cells are cultured for an additional period.

In Step (3), the recombinant polypeptide may be included in the cells, or expressed on the cell membrane, or secreted out of the cell. If desired, physical, chemical and other properties can be utilized in various isolation methods to isolate and purify the recombinant protein. These methods are well-known to those skilled in the art and include, but are not limited to conventional renaturation treatment, treatment by a protein precipitant (such as salt precipitation), centrifugation, cell lysis by osmosis, sonication, supercentrifugation, molecular sieve chromatography or gel chromatography, adsorption chromatography, ion exchange chromatography, HPLC, and any other liquid chromatography, and a combination thereof.

The polypeptide of the invention and its antagonist, agonist and inhibitor can be used for treating diseases such as various malignant tumors, adrenoprival disease, dermatitis, inflammation, HIV infection and immune system diseases directly.

XAP-2 is a transcription factor in the process of organism development and plays an important role in the gene expression regulation of organism early development. The abnormal expression of this protein in human body will lead to various genes' abnormal expression and cause related diseases.

The polypeptide in this invention belongs to transcription factor XAP-2 proteins. It has the characteristic sequence of this protein family and shares similar biologic function with them. The abnormal expression of this polypeptide in human body will lead to abnormal expression of genes and abnormal synthesis of proteins, and thus cause related diseases, these diseases include but not limited to:

Common embryogenesis malformation, like head-face dysgenesis, synpodia malformation, dysostosis, definitive operation for Hirschsprung disease, polydactyly malformation et al.

Various common tumors, like skin epidermoid carcinoma, osseous tumour(sarcom), various leukemia, (malignancy)glial cell tumor, (malignancy) hydatidiform mole, (malignancy)teratoma et al.

The metabolism disorder of proteins will mainly affect their following physiological functions and then lead to related diseases. These diseases include but not limited to:

Provide energy for organisms, maintain tissue growth, renew, and repair:

Amyotrophy, limbs feeble, body emaciation, and even dyscrasia.

Produce physiologic actively substances, like hormone, antibody, and amines et al.

The disfunction of peptide hormone will lead to the following diseases:

Insulin and glucagons: diabetes mellitus, hypoglycemia et al.

Hypothalamic and pituitary hormone: giantism, dwarfism, acromegaly, Cushing's syndrome, primary hyperaldosteronism, chronicity hypoadrenocorticism, hyperthyroidism, hypothyroidism(cretinism, Juvenile hypothyroidism, adult hypothyroidism,), man/woman infertility, menoxenia(functional metrorrhagia, menoschesis, polycystic ovary syndrome, premenstrual tension syndrome, climacteric syndrome), sex developmental disorder, diabetes insipidus, incorrect antidiuretic secretion syndrome, lactating abnormality et al.

Parathyroid hormone: hyperparathyroidism, hypoparathyroidism et al.

Gastrointestinal tract hormone: peptic ulcer, chronic dyspepsia, chronic gastritis et al.

Amines metabolism disorder can lead to the following diseases:

Arrhythmia, shock, abalienation, epilepsy, tarantism, hepatic coma (levarterenol, Υ-amino butyric acid,5-serotonin, glutamine), motion sickness, I-allergic disease (urticaria, pollinosis, allergic rhinitis, allergic skin), peptic ulcer(histamin), hypercholesterolemia (taurine), tumor(polyamine) et al;

Antibody deficiency will lead to various infection:

Hematosepsis, acute bacterial meningitis, pneumonia, tracheitis, otitis media, pyodermia et al.

The specific physiologic activity of some proteins.

Various hemoglobinopathy(anemia, icterus, tissue hypoxia caused organic acidemia), various coagulation factors deficiency diseases(bleed), muscular spasm, myotonus, myoparalysis (actin), hyperlipoproteinemia et al.

According to the above-mentioned, the polypeptide in this invention and its agonist, atagonist, and inhibitor can be directly used for treatment of various diseases, like embryogenesis malformation, tumor, diabetes mellitus, menoxenia, peptic ulcer, arrhythmia, anemia and epilepsy.

The invention also provides methods for screening compounds so as to identify an agent which enhances transcription factor XAP-2 protein 49.72 activity (agonists) or decrease transcription factor XAP-2 protein 49.72 activity (antagonists). The agonists enhance the biological functions of transcription factor XAP-2 protein 49.72 such as inactivation of cell proliferation, while the antagonists prevent and cure the disorders associated with the excess cell proliferation, such as various cancers. For example, in the presence of an agent, the mammal cells or the membrane preparation expressing transcription factor XAP-2 protein 49.72 can be incubated with the labeled transcription factor XAP-2 protein 49.72 to determine the ability of the agent to enhance or inhibit the interaction.

Antagonists of transcription factor XAP-2 protein 49.72 include antibodies, compounds, receptor deletants and analogues. The antagonists of transcription factor XAP-2 protein 49.72 can bind to transcription factor XAP-2 protein 49.72 and eliminate or reduce its function, or inhibit the production of transcription factor XAP-2 protein 49.72, or bind to the active site of said polypeptide so that the polypeptide can not function biologically.

When screening for compounds as an antagonist, transcription factor XAP-2 protein 49.72 may be added into a biological assay. It can be determined whether the compound is an antagonist or not by determining its effect on the interaction between transcription factor XAP-2 protein 49.72 and its receptor. Using the same method as that for screening compounds, receptor deletants and analogues acting as antagonists can be selected. Polypeptide molecules capable of binding to transcription factor XAP-2 protein 49.72 can be obtained by screening a polypeptide library comprising various combinations of amino acids bound onto a solid matrix. Usually, transcription factor XAP-2 protein 49.72 is labeled in the screening.

The invention further provides a method for producing antibodies using the polypeptide, and its fragment, derivative, analogue or cells as an antigen. These antibodies may be polyclonal or monoclonal antibodies. The invention also provides antibodies against epitopes of transcription factor XAP-2 protein 49.72. These antibodies include, but are not limited to, polyclonal antibody, monoclonal antibody, chimeric antibody, single-chain antibody, Fab fragment and the fragments produced by a Fab expression library.

Polyclonal antibodies can be prepared by immunizing animals, such as rabbit, mouse, and rat, with transcription factor XAP-2 protein 49.72. Various adjuvants, including but are not limited to Freund's adjuvant, can be used to enhance the immunization. The techniques for producing transcription factor XAP-2 protein 49.72 monoclonal antibodies include, but are not limited to, the hybridoma technique (Kohler and Milstein. Nature, 1975, 256: 495-497), the trioma technique, the human B-cell hybridoma technique, the EBV-hybridoma technique and so on. A chimeric antibody comprising a constant region of human origin and a variable region of non-human origin can be produced using methods well-known in the art (Morrison et al, PNAS, 1985, 81: 6851). Furthermore, techniques for producing a single-chain antibody (U.S. Pat. No. 4,946,778) are also useful for preparing single-chain antibodies against transcription factor XAP-2 protein 49.72.

The antibody against transcription factor XAP-2 protein 49.72 can be used in immunohistochemical method to detect the presence of transcription factor XAP-2 protein 49.72 in a biopsy specimen.

The monoclonal antibody specific to transcription factor XAP-2 protein 49.72 can be labeled by radioactive isotopes, and injected into human body to trace the location and distribution of transcription factor XAP-2 protein 49.72. This radioactively labeled antibody can be used in the non-wounding diagnostic method for the determination of tumor location and metastasis.

Antibodies can also be designed as an immunotoxin targeting a particular site in the body. For example, a monoclonal antibody having high affinity to transcription factor XAP-2 protein 49.72 can be covalently bound to bacterial or plant toxins, such as diphtheria toxin, ricin, ormosine. One common method is to challenge the amino group on the antibody with sulfydryl cross-linking agents, such as SPDP, and bind the toxin onto the antibody by interchanging the disulfide bonds. This hybrid antibody can be used to kill transcription factor XAP-2 protein 49.72-positive cells.

The antibody of the invention is useful for the therapy or the prophylaxis of disorders related to the transcription factor XAP-2 protein 49.72. The appropriate amount of antibody can be administrated to stimulate or block the production or activity of transcription factor XAP-2 protein 49.72.

The invention further provides diagnostic assays for quantitative and in situ measurement of transcription factor XAP-2 protein 49.72 level. These assays are well known in the art and include FISH assay and radioimmunoassay. The level of transcription factor XAP-2 protein 49.72 detected in the assay can be used to illustrate the importance of transcription factor XAP-2 protein 49.72 in diseases and to determine the diseases associated with transcription factor XAP-2 protein 49.72.

The polypeptide of the invention is useful in the analysis of polypeptide profile. For example, the polypeptide can be specifically digested by physical, chemical, or enzymatic means, and then analyzed by one, two or three dimensional gel electrophoresis, preferably by spectrometry.

Transcription factor XAP-2 protein 49.72 polynucleotides also have many therapeutic applications. Gene therapy technology can be used in the therapy of abnormal cell proliferation, development or metabolism, which are caused by the loss of transcription factor XAP-2 protein 49.72 expression or the abnormal or non-active expression of transcription factor XAP-2 protein 49.72. Recombinant gene therapy vectors, such as virus vectors, can be designed to express mutated transcription factor XAP-2 protein 49.72 so as to inhibit the activity of endogenous transcription factor XAP-2 protein 49.72. For example, one form of mutated transcription factor XAP-2 protein 49.72 is a truncated transcription factor XAP-2 protein 49.72 whose signal transduction domain is deleted. Therefore, this mutated transcription factor XAP-2 protein 49.72 can bind the downstream substrate without the activity of signal transduction. Thus, the recombinant gene therapy vectors can be used to cure diseases caused by abnormal expression or activity of transcription factor XAP-2 protein 49.72. The expression vectors derived from a virus, such as retrovirus, adenovirus, adeno-associated virus, herpes simplex virus, parvovirus, and so on, can be used to introduce the transcription factor XAP-2 protein 49.72 gene into the cells. The methods for constructing a recombinant virus vector harboring transcription factor XAP-2 protein 49.72 gene are described in the literature (Sambrook, et al. supra). In addition, the recombinant transcription factor XAP-2 protein 49.72 gene can be packed into liposome and then transferred into the cells.

The methods for introducing the polynucleotides into tissues or cells include directly injecting the polynucleotides into tissue in the body; or introducing the polynucleotides into cells in vitro with vectors, such as virus, phage, or plasmid, etc, and then transplanting the cells into the body.

Also included in the invention are ribozyme and the oligonucleotides, including antisense RNA and DNA, which inhibit the translation of the transcription factor XAP-2 protein 49.72 mRNA. Ribozyme is an enzyme-like RNA molecule capable of specifically cutting certain RNA. The mechanism is nucleic acid endo-cleavage following specific hybridization of ribozyme molecule and the complementary target RNA. Antisense RNA and DNA as well as ribozyme can be prepared by using any conventional techniques for RNA and DNA synthesis, e.g., the widely used solid phase phosphite chemical method for oligonucleotide synthesis. Antisense RNA molecule can be obtained by the in vivo or in vitro transcription of the DNA sequence encoding said RNA, wherein said DNA sequence is integrated into the vector and downstream of the RNA polymerase promoter. In order to increase its stability, a nucleic acid molecule can be modified in many manners, e.g., increasing the length of two the flanking sequences, replacing the phosphodiester bond with the phosphothioester bond in the oligonucleotide.

The polynucleotide encoding transcription factor XAP-2 protein 49.72 can be used in the diagnosis of transcription factor XAP-2 protein 49.72 related diseases. The polynucleotide encoding transcription factor XAP-2 protein 49.72 can be used to detect whether transcription factor XAP-2 protein 49.72 is expressed or not, and whether the expression of transcription factor XAP-2 protein 49.72 is normal or abnormal in the case of diseases. For example, transcription factor XAP-2 protein 49.72 DNA sequences can be used in the hybridization with biopsy samples to determine the expression of transcription factor XAP-2 protein 49.72. The hybridization methods include Southern blotting, Northern blotting and in situ blotting, etc., which are well-known and established techniques. The corresponding kits are commercially available. A part of or all of the polynucleotides of the invention can be used as probe and fixed on a microarray or DNA chip for analysis of differential expression of genes in tissues and for the diagnosis of genes. The transcription factor XAP-2 protein 49.72 specific primers can be used in RNA-polymerase chain reaction and in vitro amplification to detect transcripts of transcription factor XAP-2 protein 49.72.

Further, detection of mutations in transcription factor XAP-2 protein 49.72 gene is useful for the diagnosis of transcription factor XAP-2 protein 49.72 -related diseases. Mutations of transcription factor XAP-2 protein 49.72 include site mutation, translocation, deletion, rearrangement and any other mutations compared with the wild-type transcription factor XAP-2 protein 49.72 DNA sequence. The conventional methods, such as Southern blotting, DNA sequencing, PCR and in situ blotting, can be used to detect a mutation. Moreover, mutations sometimes affects the expression of protein. Therefore, Northern blotting and Western blotting can be used to indirectly determine whether the gene is mutated or not.

Sequences of the present invention are also valuable for chromosome identification. The sequence is specifically targeted to and can hybridize with a particular location on an individual human chromosome. There is a current need for identifying particular sites of gene on the chromosome. Few chromosome marking reagents based on actual sequence data (repeat polymorphism) are presently available for marking chromosomal location. The mapping of DNA to chromosomes according to the present invention is an important first step in correlating those sequences with genes associated with disease.

Briefly, sequences can be mapped to chromosomes by preparing PCR primers (preferably 15-35 bp) from the cDNA. These primers are then used for PCR screening of somatic cell hybrids containing individual human chromosomes. Only those hybrids containing the human gene corresponding to the primer will yield an amplified fragment.

PCR mapping of somatic cell hybrids is a rapid procedure for assigning a particular DNA to a particular chromosome. Using the oligonucleotide primers of the invention, sublocalization can be achieved with panels of fragments from specific chromosomes or pools of large genomic clones in an analogous manner. Other mapping strategies that can similarly be used to map to its chromosome include in situ hybridization, prescreening with labeled flow-sorted chromosomes and preselection by hybridization to construct chromosome specific-cDNA libraries.

Fluorescence in situ hybridization (FISH) of a cDNA clones to a metaphase chromosomal spread can be used to provide a precise chromosomal location in one step. For a review of this technique, see Verma et al., Human Chromosomes: a Manual of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis.

Next, it is necessary to determine the differences in the cDNA or genomic sequence between affected and unaffected individuals. If a mutation is observed in some or all of the affected individuals but not in any normal individuals, then the mutation is likely to be the cause of the disease. Comparison of affected and unaffected individuals generally involves first looking for structural alterations in the chromosomes, such as deletions or translocations, that are visible from chromosome level, or detectable using PCR based on that DNA sequence. With current resolution of physical mapping and genetic mapping techniques, a cDNA precisely localized to a chromosomal region associated with the disease could be one of 50 to 500 potential causative genes. (This assumes 1 megabase mapping resolution and one gene per 20 kb).

According to the invention, the polypeptides, polynucleotides and its mimetics, agonists, antagonists and inhibitors may be employed in combination with a suitable pharmaceutical carrier. Such a carrier includes but is not limited to water, glucose, ethanol, salt, buffer, glycerol, and combinations thereof. Such compositions comprise a safe and effective amount of the polypeptide or antagonist, as well as a pharmaceutically acceptable carrier or excipient with no influence on the effect of the drug. These compositions can be used as drugs in disease treatment.

The invention also provides a pharmaceutical pack or kit comprising one or more containers filled with one or more of the ingredients of the pharmaceutical compositions of the invention. With such container(s) there may be a notice from a governmental agency, that regulates the manufacture, use or sale of pharmaceuticals or biological products, the notice reflects government's approval for the manufacture, use or sale for human administration. In addition, the polypeptides of the invention may be employed in conjunction with other therapeutic compounds.

The pharmaceutical compositions may be administered in a convenient manner, such as through topical, intravenous, intraperitoneal, intramuscular, subcutaneous, intranasal or intradermal routes. transcription factor XAP-2 protein 49.72 is administered in an amount, which is effective for treating and/or prophylaxis of the specific indication. The amount of transcription factor XAP-2 protein 49.72 administrated on patient will depend upon various factors, such as delivery methods, the subject's health, the judgment of the skilled clinician.

### EXAMPLES

The invention is further illustrated by the following examples. It is appreciated that these examples are only intended to illustrate the invention, not to limit the scope of the invention. For the experimental methods in the following examples, they are performed under routine conditions, e.g., those described by Sambrook. et al., in Molecule Clone: A Laboratory Manual, New York: Cold Spring Harbor Laboratory Press, 1989, or as instructed by the manufacturers, unless otherwise specified.

### Example 1

### Cloning of Transcription Factor XAP-2 Protein 49.72 Gene

Total RNA from a human embryonic brain was extracted by the one-step method with guanidinium isocyanate/phenol/chloroform. The poly(A) mRNA was isolated from the total RNA with Quik mRNA Isolation Kit (Qiegene). cDNA was prepared by reverse transcription with 2 μg poly(A) mRNA. The cDNA fragments were inserted into the polyclonal site of pBSK(+) vector (Clontech) using Smart cDNA cloning kit (Clontech) and then transformed into DH5α to form the cDNA library. The 5′- and 3′-ends of all clones were sequenced with Dye terminate cycle reaction sequencing kit (Perkin-Elmer) and ABI 377 Automatic Sequencer (Perkin-Elmer). The sequenced cDNA were compared with the public database of DNA sequences (Genebank) and the DNA sequence of one clone 2170e05 was found to be a novel DNA sequence. The inserted cDNA sequence of clone 2170e05 was dual-directionally sequenced with a serial of synthesized primers. It was indicated that the full length cDNA contained in clone 2170e05 was 1778 bp (<210> 1) with a 1359 bp ORF located in positions 216-1574 which encoded a novel protein (<210> 2). This clone was named pBS-2170e05 and the encoded protein was named transcription factor XAP-2 protein 49.72.

### Example 2

### Homology Search of cDNA Clone

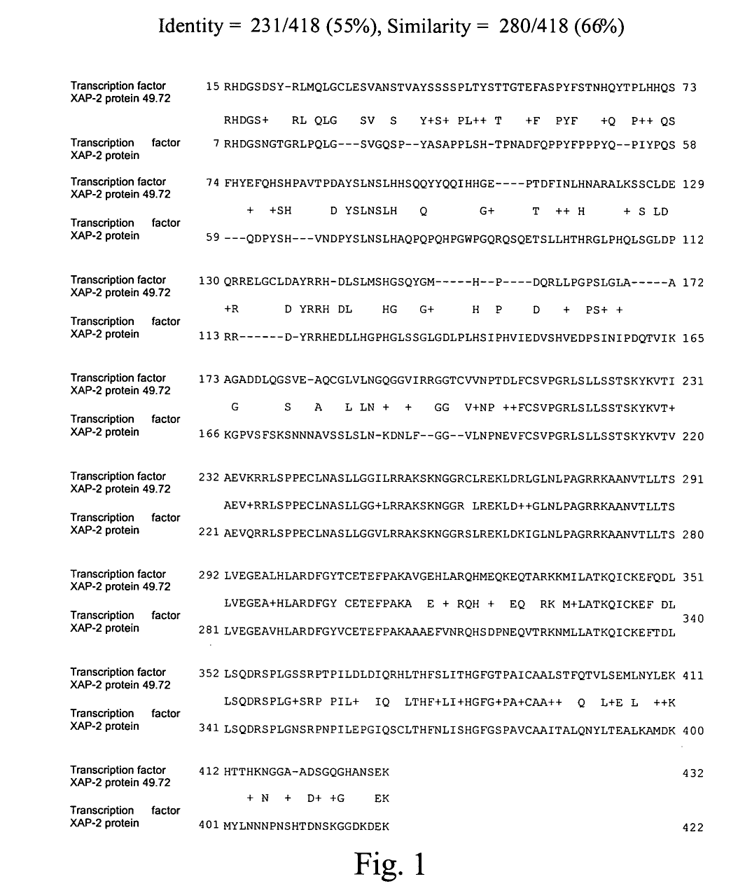

The homology research of the DNA sequence and its protein sequence of transcription factor XAP-2 protein 49.72 of the invention were performed by Blast (Basic Local Alignment Search Tool) (Altschul, S F et al.J.Mol.Biol. 1990; 215: 403-10) in databases such as Genbank, Swissport, etc. The most homologous gene to transcription factor XAP-2 protein 49.72 of the invention is known Transcription factor XAP-2 protein. The Genbank accession number of its encoded protein is M515745. The alignment result of the protein was shown in FIG. 1. Two proteins are highly homologous with an identity of 55% and a similarity of 66%.

### Example 3

### Cloning Transcription Factor XAP-2 Protein 49.72 Gene by RT-PCR

The template was total RNA extracted from a human embryonic brain. The reverse transcription was carried out with oligo-dT primer to produce cDNAs. After cDNA purified with Qiagen Kit, PCR was carried out with the following primers:

| | | |
|---|---|---|
| Primer1: | 5′-GGTGCAGAGGGAAAATTTTGTTCC-3′ | (<210>3) |
| Primer2: | 5′-CATAGGCCGAGGCGGCCGACATGT-3′ | (<210>4) |

Primer 1 is the forward sequence started from position 1 of 5′ end of <210> 1.

Primer 2 is the reverse sequence of the 3′ end of <210> 1.

The amplification condition was a 50 ul reaction system containing 50 mmol/L KCl, 10 mmol/L Tris-Cl (pH8.5), 1.5 mmol/L MgCl₂, 200 umol/L dNTP, 10 pmol of each primer, 1U Taq DNA polymerase(Clontech). The reaction was performed on a PE 9600 DNA amplifier with the following parameters: 94° C. 30 sec, 55° C. 30 sec, and 72° C. 2 min for 25 cycles. β-actin was used as a positive control, and a blank template, as a negative control in RT-PCR. The amplified products were purified with a QIAGEN kit, and linked with a pCR vector (Invitrogen) using a TA Cloning Kit. DNA sequencing results show that the DNA sequence of PCR products was identical to nucleotides 1-1778 bp of <210> 1.

### Example 4

### Northern Blotting of Expression of Transcription Factor XAP-2 Protein 49.72 Gene

Total RNA was extracted by one-step method (Anal. Biochem 1987, 162, 156-159) with guanidinium isocyanate-phenol-chloroform. That is, homogenate the organize using 4M guanidinium isocyanate-25 mM sodium citrate, 0.2M sodium acetate (pH4.0), add 1 volume phenol and ⅕ volume chloroform-isoamyl alcohol(49:1), centrifuge after mixing. Take out the water phase, add 0.8 volume isopropyl alcohol, then centrifuge the mixture. Wash the RNA precipitation using 70% ethanol, then dry, then dissolve it in the water. 20 μg RNA was electrophoresed on the 1.2% agarose gel containing 20 mM 3-(N-morpholino) propane sulfonic acid(pH 7.0)-5 mM sodium acetate-imM EDTA-2.2M formaldehyde. Then transfer it to a nitrocellulose filter. Prepare the ³²P-labelled DNA probe with α-³²P DATP by random primer method. The used DNA probe is the coding sequence (216 bp-1574 bp) of transcription factor XAP-2 protein 49.72 amplified by PCR indicated in FIG. 1. The nitrocellulose filter with the transferred RNA was hybridized with the ³²P-labelled DNA probe (2×10⁶ cpm/ml) overnight in a buffer containing 50% formamide-25 mM KH₂PO₄(Ph7.4)-5×Denhardt's solution and 200 μg/ml salmine. Then wash the filter in the 1×SSC-0.1% SDS, at 55° C., for 30 min. Then analyze and quantitative determinate using Phosphor Imager.

### Example 5

### In Vitro Expression, Isolation and Purification of Recombinant Transcription Factor XAP-2 Protein 49.72

A pair of primers for specific amplification was designed based on <210> 1 and the encoding region in FIG. 1, the sequences are as follows:

| Primer3: | 5′-CATGCTAGCATGTCAACTACCTTTCCGGGACTA-3′ |
|---|---|
| | (<210>5) |
| Primer4: | 5′-CCCGAGCTCCTAGTCTGTCTTTTCTGTTTTGCC-3′ |
| | (<210>6) |

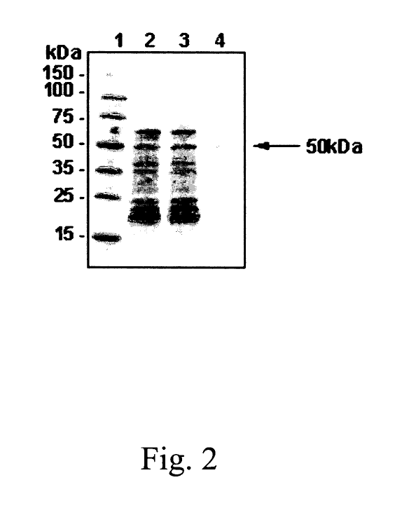

These two primers contain a NheI and SacI cleavage site on the 5′ end respectively. Within the sites are the coding sequences of the 5′ and 3′ end of the desired gene. NheI and SacI cleavage sites were corresponding to the selective cleavage sites on the expression vector pET-28b(+) (Novagen, Cat. No. 69865.3). PCR amplification was performed with the plasmid pBS-2170e05 containing the full-length target gene as a template. The PCR reaction was subject to a 50 μl system containing 10pg pBS-2170e05 plasmid, 10 pmol of Primer-3 and 10 pmol of Primer-4, 1 μl of Advantage polymerase Mix (Clontech). The parameters of PCR were 94° C. 20 sec, 60° C. 30 sec, and 68° C. 2 min for 25 cycles. After digesting amplification products and the plasmid pET-28(+) by NheI and SacI, the large fragments were recovered and ligated with T4 ligase. The ligated product was transformed into E. coli DH5α with the calcium chloride method. After cultured overnight on a LB plate containing a final concentration of 30 μg/ml kanamycin, positive clones were selected out using colony PCR and then sequenced. The positive clone (pET-2170e05) with the correct sequence was selected out and the recombinant plasmid thereof was transformed into BL21(DE3)plySs (Novagen) using the calcium chloride method. In a LB liquid medium containing a final concentration of 30 μg/ml of kanamycin, the host bacteria BL21(pET-2170e05) were cultured at 37° C. to the exponential growth phase, then IPTG were added with the final concentration of 1 mmol/L, the cells were cultured for another 5 hours, and then centrifuged to harvest the bacteria. After the bacteria were sonicated, the supernatant was collected by centrifugation. Then the purified desired protein—transcription factor XAP-2 protein 49.72 was obtained by a His.Bind Quick Cartridge (Novagen) affinity column with binding 6His-Tag. SDS-PAGE showed a single band at 49.72 kDa (FIG. 2). The band was transferred onto the PVDF membrane and the N terminal amino acid was sequenced by Edams Hydrolysis, which shows that the first 15 amino acids on N-terminus were identical to those in <210> 2.

### Example 6

### Preparation of Antibody Against Transcription Factor XAP-2 Protein 49.72

The following specific transcription factor XAP-2 protein 49.72 polypeptide was synthesized by a polypeptide synthesizer (PE-ABI): NH2-Met-Ser-Thr-Thr-Phe-Pro-Gly-Leu-Val-His-Asn-Ala-Glu-Ile-Arg-COOH (<210>7). The polypeptide was conjugated with hemocyanin and bovine serum albumin (BSA) respectively to form two composites (See Avrameas et al., Immunochemistry, 1969, 6: 43). 4 mg of hemocyanin-polypeptide composite was used to immunize rabbit together with Freund's complete adjuvant. The rabbit was re-immunized with the hemocyanin-polypeptide composite and Freund's incomplete adjuvent 15 days later. The titer of antibody in the rabbit sera was determined with a titration plate coated with 15 μg/ml BSA-polypeptide composite by ELISA. The total IgG was isolated from the sera of an antibody positive rabbit with Protein A-Sepharose. The polypeptide was bound to Sepharose 4B column activated by cyanogen bromide. The antibodies against the polypeptide were isolated from the total IgG by affinity chromatography. The immunoprecipitation approved that the purified antibodies could specifically bind to transcription factor XAP-2 protein 49.72.

### Example 7

### Application of the Polynucleotide Fragments as Hybrid Probes

Oligonucleotides selected from the polynucleotide of the instant invention can be versatilly applied as hybrid probes. The probes could be used to determine the existence of polynucleotide of the invention or its homologous polynucleotide sequences by hybridization with genomic, or cDNA libraries from normal or clinical tissues of various origins. The probes could be further used to determine whether polynucleotide of the invention or its homologous polynucleotide sequences are abnormally expressed in cells from normal or clinical tissues.

The aim of the following example is to select suitable oligonucletide fragments from <210> 1 as hybird probes to apply in membrane hybridization to determine whether there is polynucleotide of said invention or its homologous polynucleotide sequences in examined tissues. Membrane hybridization methods include dot hybridization, Southern blot, Northern blot, and replica hybridization. All these methods follow nearly the same steps after the polynucleotide samples are immobilized on membranes. These same steps are: membranes with samples immobilized on are prehybridized in hybrid buffer not containing probes to block nonspecific binding sites of the samples on membranes. Then prehybridization buffer is replaced by hybridization buffer containing labeled probes and incubation continues at the appropriate temperature so probes hybridize with the target nucleotides. Free probes are washed off by a series of washing steps after the hybridization step. A high-stringency washing condition (relatively low salt concentration and high temperature) is applied to reduce the hybridization background but retain highly specific signal. Two types of probes are selected for the example: the first type is oligonucleotides identical or annealed to <210>1 the second type is oligonucleotides partially identical or partially annealed to <210> 1. Dot blot method is applied in the said example for immobilization of the samples on membrane. The strongest specific signal is produced by hybridization between first type probes and samples after relatively stringent membrane washing steps.

Selection of Probes

The principles below should be followed and some things should be taken into consideration for selection of oligonucleotide fragments from <210> 1 as hybrid probes:

1. The optimal length of probes should be between eighteen and fifty nucleotides.

2. GC content should be between 30% and 70%, since nonspecific hybridization increases when GC content is more than 70%.

3. There should be no complementary regions within the probes themselves.

4. Probes satisfying the requirements above could be initially selected for further computer-aided sequence analysis, which includes homology comparison between the initial selected probes and its source sequence region (<210> 1), and other known genomic sequences and their complements. Generally, probes should not be used when they share fifteen identical continuous base pairs, or 85% homology with a non-target region.

5. Whether said initial selected probes should be chosen for final application depends on further experimental confirmation.

The following two probes are selected and synthesized after the analysis above:

Probe one belongs to the first type probes, which is completely identical or annealed to the gene fragments of <210> 1(41 Nt);

5′-TGTCAACTACCTTTCCGGGACTAGTCCACAATGCCGAGATA-3′
(<210>8)

Probe two belongs to the second type probes which is a replaced or mutant sequence of the gene fragments of <210> 1, or of its complementary fragments (41 Nt):

5′-TGTCAACTACCTTTCCGGGACTAGTCCACAATGCCGAGATA-3′
(<210>9)

Any other frequently used reagents unlisted but involved in the following concrete experimental steps and their preparation methods can be found in the reference: DNA PROBES G. H. Keller; M. M. Manak; Stockton Press, 1989 (USA) or a more commonly used molecular cloning experimental handbook (Molecular Cloning) (J. Sambrook et al. Acadimic press, 1998, 2^{nd} edition)

Sample Preparation:

1) DNA Extraction from Fresh or Frozen Tissues

Steps: 1) move the fresh or newly thawed tissue (source tissue of the polyucleotide) onto a ice-incubated dish containing phosphate-buffered saline (PBS). Cut the tissue into small pieces with a scissor or an operating knife. Tissue should be kept moist through the operation. 2) mince the tissue by centrifugation at 2,000 g for 10 minutes. 3) resuspend the pellet (about 10 ml/g) with cold homogenating buffer (0.25 mol/l saccharose; 25 mmol/l Tris-HCl, pH 7.5; 25 m mol/L NaCl; 25 mmol/L MgCl₂, 4) at 4° C., and homogenate tissue suspension at full speed with an electronic homogenizer until it's completely smashed. 5) centrifuge at 1,000 g for 10 minutes. 6) resuspend the cell pellet (1-5 ml per 0.1 g initial tissue sample), and centrifuge at 1,000 g for 10 minutes. 7) resuspend the pellet with lysis buffer (1-5 ml per 0.1 g initial tissue sample), and continue to the phenol extraction method.

2) Phenol Extraction of DNA

Steps: 1) wash cells with 1-10 ml cold PBS buffer and centrifuge at 1000 g for 10 minutes. 2) resuspend the precipitated cells with at least 100 μl cold cell lysis buffer (1×10⁸ cells/ml). 3) add SDS to a final concentration of 1%. Addition of SDS into the cell precipitation before cell resuspension will cause the formation of large cell aggregates which are difficult to break and total yield will be reduced. This phenomenon is especially severe when extracting more than 10⁷ cells. 4) add protease K to the final concentration of 200 ug/ml. 5) incubate at 50° C. for an hour or shake gently overnight at 37° C. 6) add an equal volume of phenol: chloroform: isoamyl alcohol (25:24:1) to the DNA solution to be purified in a microcentrifuge tube, and centrifuge for 10 minutes. If the two phases are not clearly separated, the solution should be recentrifuged. 7) remove the water phase to a new tube. 8) add an equal volume of chloroform: isoamyl alcohol (24:1) and centrifuge for 10 minutes. 9) remove the water phase containing DNA to a new tube and then purify DNA by ethanol precipitation.

3) DNA Purification by Ethanol Precipitation

Steps: 1) add 1/10 vol of 2 mol/L sodium acetate and 2 vol of cold 100% ethanol into the DNA solution, mix and place at −20° C. for an hour or overnight. 2) centrifuge for 10 minutes. 3) carefully spill the ethanol. 4) add 500 μl of cold 70% ethanol to wash the pellet and centrifuge for 5 minutes. 5) carefully spill the ethanol, add 500 ul cool ethanol to wash the pellets and centrifuge for 5 minutes. 6) carefully remove the ethanol and invert the tube on bibulous paper to remove the remaining ethanol. Air dry for 10-15 minutes to evaporate ethanol on pellet surface. But notice not to dry the pellet completely since completely dry pellet is difficult to be dissolved again. 7) resuspend the DNA pellet with a small volume of TE or water. Spin at low speed or blow with a drip tube, and add TE gradually and mix until DNA is completely dissolved. Add about 1 μl TE for every 1-5×10⁶ cells.

The following 8-13 steps are applied only when contamination must be removed, otherwise go to step 14 directly. 8) add RNase A into DNA solution to a final concentration of 100 μg/ml and incubate at 37° C. for 30 minutes. 9) add SDS and protease K to the final concentration of 0.5% and 100 μg/ml respectively, and incubate at 37° C. for 30 minutes. 10) add an equal volume of phenol: chloroform: isoamyl alcohol (25:24:1), and centrifuge for 10 minutes. 11) carefully remove the water phase and extract it with an equal volume of chloroform: isoamyl alcohol (24:1) and centrifuge for 10 minutes. 12) carefully remove the water phase, and add 1/10 vol of 2 mol/L sodium acetate and 2.5 vol of cold 100% ethanol, then mix and place at −20° C. for an hour. 13) wash the pellet with 70% ethanol and 100% ethanol, air dry and resuspend DNA as same as steps 3-6. 14) determine the purity and production of DNA by A₂₆₀ and A₂₈₀ assay. 15) separate DNA sample into several portions and store at −20° C.

Preparation of Sample Membrane:

1) Take 4×2 pieces of nitrocellulose membrane (NC membrane) of desired size, and lightly mark out the sample dot sites and sample number with a pencil. Every probe needs two pieces of NC membrane, so then membranes could be washed under high stringency condition and stringency condition individually in the following experimental steps.

2) Pipette 15 μl of samples and control individually, dot them on the membrane, and dry at room temperature.

3) Place the membranes on filter paper soaked in 0.1 mol/l NaOH, 1.5 mol/L NaCl, leave for 5 minutes (twice), and allow to dry. Transfer the membranes on filter paper soaked in 0.5 mol/L Tris-HCl (pH7.0), 3 mol/L NaCl, leave for 5 minutes (twice), and allow to dry.

4) Place the membranes between clean filter paper, packet with aluminum foil, and vacuum dry at 60-80° C. for 2 hours.

Labeling of Probes

1) Add 3 μl probe (0.1OD/10 μl), 2 μl kinase buffer, 8-10 μCi γ-³²P-dATP+2U Kinase, and add water to the final volume of 20 μl.

2) Incubate at 37° C. for 2 hours.

3) Add ⅕ vol bromophenol blue indicator (BPB).

4) Load that sample on Sephadex G-50 column.

5) Collect the first peak before the elution of ³²P-Probe (monitor the eluting process by Monitor).

6) Five drops each tube and collect for 10-15 tubes.

7) Measure the isotope amount with liquid scintillator

8) Merged collection of the first peak is the prepared ³²P-Probe (the second peak is free γ-³²P-DATP)

Prehybridization

Place the sample membranes in a plastic bag, add 3-10 mg prehybrid buffer (10×Denhardt×s; 6×SSC, 0.1 mg/ml CT DNA (calf thymus gland DNA)), seal the bag, and shake on a 68° C. water bath for two hours hybridization.

Hybridization

Cut off a corner of the plastic bag, add in prepared probes, seal the bag, and shake on a 42° C. water bath overnight.

Membrane Washing Under a High-Stringency Condition:

1) Take out the hybridized sample membranes

2) Wash the membranes with 2×SSC, 0.1% SDS at 40° C. for 15 minutes (twice).

3) Wash the membranes with 0.1×SSC, 0.1% SDS at 40° C. for 15 minutes (twice).

4) Wash the membranes with 0.1×SSC, 0.1% SDS at 55° C. for 30 minutes (twice), and dry at room temperature.

Membrane Washing Under a Low-Stringency Condition:

1) Take out the hybridized sample membranes.

2) Wash the membranes with 2×SSC, 0.1% SDS at 37° C. for 15 minutes (twice).

3) Wash the membranes with 0.1×SSC, 0.1% SDS at 37° C. for 15 minutes (twice).

4) Wash the membranes with 0.1×SSC, 0.1% SDS at 40° C. for 15 minutes (twice), and dry at room temperature.

X Ray Autoradiography:

X ray autoradiograph at −70° C. (autoradiograph time varies according to radioactivity of the hybrid spots)

Experimental Results:

In hybridization experiments carried out under low-stringency membrane washing condition, the radioactivity of the above two probes hybridization spots show no obvious difference; while in hybridization experiments carried out under high-stringency membrane washing condition, radioactivity of the hybrid spot by probe one is obviously stronger than the other three's. So probe one could be applied in qualitative and quantitative analysis of the existence and differential expression of said invented polynucleotide in different tissues.

1-18. (Cancelled)

19. An isolated polypeptide having an Transcription factor XAP-2 protein 49.72 activity and comprising an amino acid sequence of <210> 2, or its active fragments, analogues or derivatives.

20. An isolated polypeptide having an Transcription factor XAP-2 protein 49.72 activity and comprising an amino acid sequence that is at least 95% identical to <210> 2.

21. The polypeptide of claim 20 wherein said polypeptide comprises the amino acid sequence of <210> 2.

22. An isolated polynucleotide selected from the group consisting of:
(a) a polynucleotide encoding a polypeptide that has an Transcription factor XAP-2 protein 49.72 activity and is at least 95% identical to <210> 2; or
(b) a polynucleotide complementary to polynucleotide (a).

23. A polynucleotide of claim 22 wherein the polynucleotide encodes an amino acid sequence of <210>2.

24. A polynucleotide of claim 22 wherein the sequence of said polynucleotide comprises position 216-1574 of <210>1.

25. A polynucleotide of claim 24 wherein the sequence of said polynucleotide comprises position 1-1778 of <210>1.

26. A recombinant vector comprising a polynucleotide of claim 22, and a suitable regulatory element.

27. A genetically engineered host cell comprising a polynucleotide of claim 22.

28. A method for producing a polypeptide having an activity of an Transcription factor XAP-2 protein 49.72 which comprises the steps of:
(a) culturing an engineered host cell of claim 27 under conditions suitable for the expression of an Transcription factor XAP-2 protein 49.72; and
(b) isolating a polypeptides having the activity of an Transcription factor XAP-2 protein 49.72 from the culture.

29. An antibody which binds specifically for a polypeptide of claim 20.

30. A compound simulating, enhancing, antagonizing, or inhibiting an Transcription factor XAP-2 protein 49.72 activity of a polypeptide of claim 20.

31. A compound of claim 30 which is an antisense sequence of the polynucleotide sequence of <210> 1 or its fragment which inhibits expression of <210>1 in a cell.

32. A method for regulating the activity of an Transcription factor XAP-2 protein 49.72 in vivo or in vitro, comprising applying a compound of claim 30 to an Transcription factor XAP-2 protein 49.72 or a polynucleotide encoding an Transcription factor XAP-2 protein 49.72.

33. A method for detecting a disease related to the polypeptide of claim 20, or for determining a susceptibility of a mammal thereto, said method comprising detecting the amount of expression of said polypeptide, or detecting the activity of said polypeptide, or detecting the nucleotide variant of a polynucleotide encoding the polypeptide which variant causes said abnormal expression or activity.

34. A method for screening mimetics, agonists, antagonists or inhibitors of an Transcription factor XAP-2 protein 49.72; or for peptide profiling, the method comprising labeling a polypeptide according to claim 20 and applying the labeled polypeptide in the screening or profiling.

35. A method for nucleic acid amplification, or for nucleic acid hybridization reaction, or for manufacture of gene chip or microarray, the method comprising synthesizing a polynucleotide of claim 22.

36. A pharmaceutical composition comprising a polypeptide according to claim 20, and a pharmaceutically acceptable carrier.

37. A method for the treatment of embryogenesis malformation, tumor, diabetes mellitus, menoxenia, peptic ulcer, arrhythmia, anemia and epilepsy wherein the method comprising administering an effective amount of the pharmaceutical composition of claim 36 to a patient in need thereof.


SEQUENCE LISTING (11 sequences)

SEQ ID NO: 1 - DNA, 1778 bp, Homo sapiens
  CDS (216)..(1574)
ggtgcagagggaaaattttgttcctacaggtttttaagtgggtacgaggcaaggagctatctgatccgggcaagaccattacaatttagatatctacctatagaacattttttttttcctttaaaaattggaaaatacaagaagtttggatttttttttcccgattctttttttggagggggaaattgcatcgtaagctttcggagaaacccaacatgtcaactacctttccgggactagtccacaatgccgagatacgtcacgacggatcagacagctaccgtttgatgcagcttggctgtctggagtcagtagccaattccactgtcgcctattcctcctcctctcctttaacttactccaccaccggcaccgagtttgcgtccccctacttctccactaaccaccagtacaccccgctccaccaccagtccttccattacgagtttcagcacagccacccggccgtcacccccgacgcctactctctgaactctctccaccactcgcaacagtactaccagcagatccaccacggggagcccaccgactttattaacctgcacaatgcgcgggcgctcaagtcgtcctgcctggacgagcagaggcgggagctgggctgcctcgatgcctaccgccgccatgacctgtccctcatgagccatggctctcagtatggaatgcacccagatcaaagactcctgccagggcccagcctggggctggccgccgcgggagcagacgacttgcagggctctgtggaggcccagtgtgggcttgttctcaatggccaaggtggagtgataagaagaggtggcacctgtgtggtcaaccccacagacttattttgctctgtccctggccgtttgtcccttcttagttctacttccaaatacaaggtgaccattgctgaggtaaagaggcgcctctccccacctgagtgcctcaatgcttcactcttgggaggcattttgagaagagcaaaatcaaagaatgggggccgctgcctgagagagaaattggataggcttggcttaaacttaccagcaggaagacggaaagcagctaatgtcaccctccttacttccttggttgaaggggaggctttgcacttggctcgggattttggctacacttgtgaaacagagtttccagccaaagcagtaggagaacatcttgccagacaacatatggaacagaaagaacagacagcaagaaaaaagatgatcctggcgaccaaacaaatctgtaaagaattccaagacctcttgagccaagatagatcaccactgggatcctccagacccactccaattctagaccttgacatccagagacatttaacacatttcagtttgatcactcatggctttgggactccggcaatatgtgcagctctaagcactttccaaacagttctcagtgaaatgctgaactacttggaaaaacacactactcacaagaacggcggagcggcggattctggccaaggacatgccaactcggagaaagctcccctgcggaaaacttcagaggctgccgtgaaagagggcaaaacagaaaagacagactagctacatcaaacagaatctatttccagagagtcttgctgctgatattttttctaatatatatatcattgagggtgactaatcttcagtggaccaaatctctacccttccccaaccctccataaaaaaacaaaaatggaaatgaaaaatgaaacaaaaaaggacaaaaccaaaaaaaaaaaaacatgtcggccgcctcggcctatg

SEQ ID NO: 2 - protein, 452 aa, Homo sapiens
MSTTFPGLVHNAEIRHDGSDSYRLMQLGCLESVANSTVAYSSSSPLTYSTTGTEFASPYFSTNHQYTPLHHQSFHYEFQHSHPAVTPDAYSLNSLHHSQQYYQQIHHGEPTDFINLHNARALKSSCLDEQRRELGCLDAYRRHDLSLMSHGSQYGMHPDQRLLPGPSLGLAAAGADDLQGSVEAQCGLVLNGQGGVIRRGGTCVVNPTDLFCSVPGRLSLLSSTSKYKVTIAEVKRRLSPPECLNASLLGGILRRAKSKNGGRCLREKLDRLGLNLPAGRRKAANVTLLTSLVEGEALHLARDFGYTCETEFPAKAVGEHLARQHMEQKEQTARKKMILATKQICKEFQDLLSQDRSPLGSSRPTPILDLDIQRHLTHFSLITHGFGTPAICAALSTFQTVLSEMLNYLEKHTTHKNGGAADSGQGHANSEKAPLRKTSEAAVKEGKTEKTD

SEQ ID NO: 3 - DNA, 24 bp, Homo sapiens
ggtgcagagggaaaattttgttcc

SEQ ID NO: 4 - DNA, 24 bp, Homo sapiens
cataggccgaggcggccgacatgt

SEQ ID NO: 5 - DNA, 33 bp, Homo sapiens
catgctagcatgtcaactacctttccgggacta

SEQ ID NO: 6 - DNA, 33 bp, Homo sapiens
cccgagctcctagtctgtcttttctgttttgcc

SEQ ID NO: 7 - protein, 15 aa, Homo sapiens
MSTTFPGLVHNAEIR

SEQ ID NO: 8 - DNA, 41 bp, Homo sapiens
tgtcaactacctttccgggactagtccacaatgccgagata

SEQ ID NO: 9 - DNA, 41 bp, Homo sapiens; identical to SEQ ID NO: 8

SEQ ID NO: 10 - protein, 416 aa, Xenopus sp.
RHDGSNGTGRLPQLGSVGQSPYASAPPLSHTPNADFQPPYFPPPYQPIYPQSQDPYSHVNDPYSLNSLHAQPQPQHPGWPGQRQSQETSLLHTHRGLPHQLSGLDPRRDYRRHEDLLHGPHGLSSGLGDLPLHSIPHVIEDVSHVEDPSINIPDQTVIKKGPVSFSKSNNNAVSSLSLNKDNLFGGVLNPNEVFCSVPGRLSLLSSTSKYKVTVAEVQRRLSPPECLNASLLGGVLRRAKSKNGGRSLREKLDKIGLNLPAGRRKAANVTLLTSLVEGEAVHLARDFGYVCETEFPAKAAAEFVNRQHSDPNEQVTRKNMLLATKQICKEFTDLLSQDRSPLGNSRPNPILEPGIQSCLTHFNLISHGFGSPAVCAAITALQNYLTEALKAMDKMYLNNNPNSHTDNSKGGDKDEK

SEQ ID NO: 11 - protein, 6 aa, Artificial Sequence
  Description of Artificial Sequence Synthetic 6X-His tag
HHHHHH